(19) United States
(12) Patent Application Publication
(10) Pub. No.: US 2023/0001394 A1
(43) Pub. Date: Jan. 5, 2023

(54) CATALYST FOR CARBONYLATION OF DIMETHYL ETHER AND METHOD FOR PREPARING THE SAME

(71) Applicant: RESEARCH & BUSINESS FOUNDATION SUNGKYUNKWAN UNIVERSITY, Suwon-si (KR)

(72) Inventors: Jong Wook BAE, Suwon-si (KR); Jong Jin KIM, Suwon-si (KR)

(73) Assignee: RESEARCH & BUSINESS FOUNDATION SUNGKYUNKWAN UNIVERSITY, Suwon-si (KR)

(21) Appl. No.: 17/857,528

(22) Filed: Jul. 5, 2022

(30) Foreign Application Priority Data
Jul. 2, 2021 (KR) 10-2021-0087018

(51) Int. Cl. B01J 29/65; B01J 31/06; B01J 31/26; B01J 35/02; B01J 35/00; B01J 37/04; B01J 37/00; B01J 37/02; B01J 37/08; B01J 29/90; B01J 38/04; C07C 67/37

(52) U.S. Cl. CPC B01J 29/65; B01J 31/06; B01J 31/26; B01J 35/026; B01J 35/0026; B01J 37/04; B01J 37/0018; B01J 37/0221; B01J 37/0219; B01J 37/0236; B01J 37/082; B01J 29/90; B01J 38/04; C07C 67/37; B01J 2531/002; B01J 2231/4288

(57) ABSTRACT

Disclosed are a catalyst for carbonylation of dimethyl ether that has high catalyst activity and can be regenerated using a fluidized bed reactor, and a method for preparing the same. The catalyst for carbonylation of dimethyl ether includes a support having a first density; and ferrierite zeolite catalyst particles bound to a surface of the support via a polymer binder and having a second density smaller than the first density.

### CROSS-REFERENCE TO RELATED APPLICATION

This application claims a benefit under 35 U.S.C § 119(a) of Korean Patent Application No. 10-2021-0087018 filed on Jul. 2, 2021, on the Korean Intellectual Property Office, the entire disclosure of which is incorporated herein by reference for all purposes.

### BACKGROUND

### 1. Field

The present disclosure relates to a catalyst for carbonylation of dimethyl ether and a method for preparing the same. More specifically, the present disclosure relates to a catalyst for carbonylation of dimethyl ether that may have high catalyst activity and may be regenerated using a fluidized bed reactor and a method for preparing the same.

### 2. Description of Related Art

Due to depletion of fossil fuels, production of alternative energy is becoming increasingly important. In this regard, the most promising material is ethanol. Ethanol may be produced in a variety of ways. In particular, a multi-step reaction scheme of synthesizing ethanol using syngas including carbon monoxide and carbon dioxide which are by-product gases of factories and steel mills is attracting attention.

This multi-step reaction scheme is eco-friendly because the scheme may regenerate harmful gases such as carbon monoxide and carbon dioxide, which is a greenhouse gas, and may effectively synthesize ethanol, and is evaluated as a very economical process due to various uses of ethanol. In this regard, a multi-step ethanol production reaction using syngas is as follows.

(Reaction 1) Synthesis of dimethyl ether (DME synthesis)

2CO+4H₂->CH₃OCH₃+H₂O

(Reaction 2) Carbonylation of dimethyl ether (DME carbonylation)

CO+CH₃OCH₃->CH₃COOCH₃

(Reaction 3) Hydrogenation of methyl acetate (MA hydrogenation)

CH₃COOCH₃+H₂->CH₃CH₂OH+CH₃OH

The most essential reaction in the above-described three-step reaction is the carbonylation of dimethyl ether, which is the second reaction. This is because, in general, when a zeolite-based catalyst is used as a catalyst for the above reaction, coke specific to zeolite is produced, and deposition of coke leads to deactivation of the catalyst. Therefore, solving the problem of Reaction 2 rather than Reaction 1 and Reaction 3 which occur relatively easily, is the most important issue in the commercialization of the scheme.

In previous studies on carbonylation of dimethyl ether, mordenite zeolite has been mainly used. However, when mordenite zeolite is used, coke is easily produced due to its structural characteristics, and deactivation of the catalyst occurs very quickly.

When ferrierite zeolite is used, coke production may be smaller than that when mordenite zeolite is used, such that the deactivation of the catalyst is suppressed to some extent. However, ferrierite zeolite reported to date does not completely suppress the deactivation. For this reason, research to regenerate the deactivated catalyst is being actively conducted.

In general, a method using a fluidized bed reactor is widely used to regenerate the catalyst. When ferrierite zeolite is used, a density thereof is too small and thus flow does not occur. Thus, a method to increase the density includes pellet preparation via extrusion and compression, and particle size adjustment via a spray method which inevitably lead to use of a special apparatus, thereby causing infrastructure-related and economic problems.

### SUMMARY

This Summary is provided to introduce a selection of concepts in a simplified form that are further described below in the Detailed Description. This Summary is not intended to identify all key features or essential features of the claimed subject matter, nor is it intended to be used alone as an aid in determining the scope of the claimed subject matter.

One purpose of the present disclosure is to provide a catalyst for carbonylation of dimethyl ether that may have high catalyst activity and may be regenerated via a fluidized bed reactor and a method for preparing the same.

Another purpose of the present disclosure is to provide a method for preparation of methyl acetate with improved productivity using the catalyst.

Another purpose of the present disclosure is to provide a method for regenerating the catalyst that has been deactivated.

Purposes in accordance with the present disclosure are not limited to the above-mentioned purpose. Other purposes and advantages in accordance with the present disclosure as not mentioned above may be understood from following descriptions and more clearly understood from embodiments in accordance with the present disclosure. Further, it will be readily appreciated that the purposes and advantages in accordance with the present disclosure may be realized by features and combinations thereof as disclosed in the claims.

A first aspect of the present disclosure provides a catalyst for carbonylation of dimethyl ether, the catalyst comprising: a support having a first density; and ferrierite zeolite catalyst particles bound to a surface of the support via a polymer binder and having a second density smaller than the first density.

In one implementation of the first aspect, a surface of each of at least some of the ferrierite zeolite catalyst particles is exposed to an outside.

In one implementation of the first aspect, the catalyst has a core-shell structure in which the ferrierite zeolite catalyst particles are coated on the surface of the support.

In one implementation of the first aspect, a ratio between a weight of the support and a weight of the ferrierite zeolite catalyst particles is in a range of 2.5:1 to 10:1.

In one implementation of the first aspect, the polymer binder includes at least one selected from a group consisting of polyethylene glycol (PEG), polyvinyl alcohol (PVA) and methyl cellulose.

In one implementation of the first aspect, when the polymer binder is polyvinyl alcohol (PVA), a content of the polymer binder is in a range of 9 to 73% by weight, based on a total weight of the support and the ferrierite zeolite catalyst particles.

In one implementation of the first aspect, when the polymer binder is methyl cellulose, a content of the polymer binder is a range of 15 to 25% by weight, based on a total weight of the support and the ferrierite zeolite catalyst particles.

In one implementation of the first aspect, the polymer binder has at least one functional group selected from a group consisting of a methoxyl group (CH₃O—), a carboxyl group (—COOH), glycerate (C₃H₅O₄—) and a hydroxyl group (—OH).

In one implementation of the first aspect, a molecular weight of the polymer binder is in a range of 146000 to 186000 g/mol.

In one implementation of the first aspect, a molar ratio (Si/Al) as a ratio of a content of silicon to a content of aluminum in the ferrierite zeolite catalyst particle is in a range of 5 to 30.

In one implementation of the first aspect, a size of the support is in a range of 50 to 150 μm, wherein a size of each of the ferrierite zeolite catalyst particles is in a range of 100 nm to 1 μm.

In one implementation of the first aspect, the first density is in a range of 750 to 800 kg/m³.

A second aspect of the present disclosure provides a method for preparing a catalyst for carbonylation of dimethyl ether, the method comprising: mixing a support having a first density, ferrierite zeolite catalyst particles having a second density smaller than the first density, and a polymer binder with each other in a solvent to prepare a mixed solution; drying the mixed solution to prepare a dried mixture; and firing the dried mixture.

In one implementation of the second aspect, in the firing of the dried mixture, a portion of the polymer binder contained in the dried mixture is decomposed and removed, and the rest thereof remains therein such that a surface of each of at least some of the ferrierite zeolite catalyst particles bound to a surface of the support is exposed to an outside.

In one implementation of the second aspect, the mixing includes: a first step of mixing the support having the first density and the ferrierite zeolite catalyst particles having the second density smaller than the first density with each other to prepare a mixture; and a second step of adding and mixing the mixture of the support and the ferrierite zeolite catalyst particles to and with a polymer binder solution.

In one implementation of the second aspect, the polymer binder solution is prepared by dissolving the polymer binder in a solvent for 1 to 6 hours under conditions of pH 6 to 8, and 80 to 100° C.

In one implementation of the second aspect, in the mixing, a ratio between a weight of the support and a weight of the ferrierite zeolite catalyst particles is in a range of 2.5:1 to 10:1.

In one implementation of the second aspect, in the mixing, a content of the polymer binder is in a range of 9 to 73% by weight, based on a total weight of the support and the ferrierite zeolite catalyst particles.

In one implementation of the second aspect, the polymer binder includes at least one selected from a group consisting of polyethylene glycol (PEG), polyvinyl alcohol (PVA) and methyl cellulose.

In one implementation of the second aspect, when the polymer binder is polyethylene glycol (PEG) or polyvinyl alcohol (PVA), the firing is performed at a temperature of 230 to 550° C. under a nitrogen atmosphere.

In one implementation of the second aspect, when the polymer binder is methyl cellulose, the firing includes: a first step of performing heat treatment at a temperature of 210 to 250° C. under a nitrogen atmosphere; a second step of performing heat-treatment at a temperature of 230 to 270° C. under a nitrogen atmosphere after the first step; and a third step of performing heat treatment at a temperature of 130 to 170° C. under a nitrogen atmosphere after the second step.

In one implementation of the second aspect, the polymer binder has at least one functional group selected from a group consisting of a methoxyl group (CH₃O—), a carboxyl group (—COOH), glycerate (C₃H₅O₄—) and a hydroxyl group (—OH).

In one implementation of the second aspect, a molecular weight of the polymer binder is in a range of 146000 to 186000 g/mol.

A third aspect of the present disclosure provides a method for preparation of methyl acetate, the method comprising performing carbonylation of dimethyl ether using carbon monoxide under presence of the catalyst as defined above, thereby converting dimethyl ether to methyl acetate.

In one implementation of the third aspect, the performing of the carbonylation of dimethyl ether using carbon monoxide under presence of the catalyst, thereby converting dimethyl ether to methyl acetate includes reacting a mixed gas containing carbon monoxide and dimethyl ether in a fluidized bed reactor at a temperature of 200 to 240° C.

A fourth aspect of the present disclosure provides a method for regenerating a catalyst, the method comprising treating a deactivated catalyst with carbon monoxide-containing gas in a fluidized bed reactor, thereby regenerating the catalyst, wherein the catalyst includes the catalyst as defined above.

In the catalyst according to the present disclosure, the content of each of the support, the ferrierite zeolite catalyst particles, and the polymer binder may be controlled to an appropriate range such that the ferrierite zeolite catalyst particles are uniformly coated on the surface of the support while maintains crystallinity thereof. Thus, the catalyst according to the present disclosure may exhibit high activity in a conversion reaction from dimethyl ether to methyl acetate, and the deactivated catalyst may be regenerated in the fluidized bed reactor and then reused.

Further, since the catalyst according to the present disclosure has the core-shell structure and is present in a form of a particle, deactivation thereof may be prevented, and coke deposition during the catalyst reaction may be suppressed, thereby enabling a stable process.

According to the method for preparing the catalyst according to the present disclosure, during the preparation, the content of each of the support, the ferrierite zeolite catalyst particles, and the polymer binder may be controlled to an appropriate range such that the ferrierite zeolite catalyst particles may be successfully bound to the surface of the support, and a portion of the polymer binder may remain via the firing process. Thus, the catalyst may be prepared in which the surface of each of at least some of the ferrierite zeolite catalyst particles bound to the support surface is exposed to the outside.

Moreover, the method for preparing the catalyst according to the present disclosure is economical and has a fast synthesis rate because the method does not use a special mechanical apparatus, which is not the case in a conventional method for preparing a catalyst for a fluidized bed in which a density is controlled using extrusion, compression, and spraying.

In addition to the effects as described above, specific effects in accordance with the present disclosure will be described together with following detailed descriptions for carrying out the disclosure.

### BRIEF DESCRIPTION OF DRAWINGS

The patent or application file contains at least one drawing executed in color. Copies of this patent or patent application publication with color drawing(s) will be provided by the Office upon request and payment of the necessary fee.

FIG. 1 shows SEM analysis images of catalysts according to Present Examples and Comparative Examples of the present disclosure.

FIG. 2 shows the results of X-ray diffraction analysis of catalysts according to Present Examples and Comparative Examples of the present disclosure.

FIG. 3 shows results of TPD analysis using ammonia of catalysts according to Present Examples and Comparative Examples of the present disclosure.

FIGS. 4A and 4B are graphs respectively showing dimethyl ether conversions and methyl acetate selectivity in carbonylation using catalysts according to Present Examples and Comparative Examples of the present disclosure.

FIGS. 5A to 5C are graphs showing TGA analysis results of catalysts according to Present Examples of the present disclosure.

### DETAILED DESCRIPTIONS

Descriptions and details of well-known steps and elements are omitted for simplicity of the description. Furthermore, in the following detailed description of the present disclosure, numerous specific details are set forth in order to provide a thorough understanding of the present disclosure. However, it will be understood that the present disclosure may be practiced without these specific details. In other instances, well-known methods, procedures, components, and circuits have not been described in detail so as not to unnecessarily obscure aspects of the present disclosure. Examples of various embodiments are illustrated and described further below. It will be understood that the description herein is not intended to limit the claims to the specific embodiments described. On the contrary, it is intended to cover alternatives, modifications, and equivalents as may be included within the spirit and scope of the present disclosure as defined by the appended claims.

The terminology used herein is for the purpose of describing particular embodiments only and is not intended to limit the present disclosure. As used herein, the singular forms “a” and “an” are intended to include the plural forms as well, unless the context clearly indicates otherwise. It will be further understood that the terms “comprises”, “comprising”, “includes”, and “including” when used in this specification, specify the presence of the stated features, integers, operations, elements, and/or components, but do not preclude the presence or addition of one or more other features, integers, operations, elements, components, and/or portions thereof.

Unless otherwise defined, all terms including technical and scientific terms used herein have the same meaning as commonly understood by one of ordinary skill in the art to which this inventive concept belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

### Catalyst for Carbonylation of Dimethyl Ether

A catalyst for carbonylation of dimethyl ether according to one embodiment of the present disclosure includes a support having a first density, and ferrierite zeolite catalyst particles bonded to a surface of the support via a polymer binder and having a second density smaller than the first density.

The support having the first density means the support having a density set such that the support can flow in a fluidized bed reactor. For example, a commercial FCC (fluid catalytic cracking) catalyst having a density of 750 to 800 kg/m³ may be used as the support. That is, the support has the density set such that the support may flow in the fluidized bed reactor. Thus, when the catalyst according to the present disclosure is deactivated, the catalyst may be regenerated in the fluidized bed reactor.

In one embodiment, a size of the support may be in a range of 50 to 150 μm, preferably in a range of 75 to 95 μm.

The ferrierite zeolite catalyst particles act as active materials for carbonylation of dimethyl ether, are bonded to the surface of the support via the polymer binder and have a second density smaller than that of the support. In this regard, a molar ratio (Si/Al) between a content of silicon and a content of aluminum in the ferrierite zeolite catalyst particle is preferably in a range of 5 to 30. Further, the ferrierite zeolite catalyst particles may include commercially available ferrierite zeolite catalyst particles, or H-form ferrierite that is synthesized by a known seed synthesis method and then is subjected to ion exchange.

In one embodiment, a size of each of the ferrierite zeolite catalyst particles may be in a range of 100 nm and 1 μm, preferably in a range of 200 and 400 nm.

Further, a ratio between a size of the support and a size of the ferrierite zeolite catalyst particle may be in a range of 150:1 to 500:1, preferably 275:1.

In one embodiment, a surface of each of at least some of the ferrierite zeolite catalyst particles may be exposed to an outside. Increase in the exposed area of the ferrierite zeolite catalyst particles may improve the catalyst activity.

In one embodiment, the catalyst according to the present disclosure may have a core-shell structure in which the ferrierite zeolite catalyst particles are coated on the surface of the support. Therefore, when the catalyst according to the present disclosure is used for carbonylation of dimethyl ether, deactivation of the catalyst may be prevented due to the core-shell structure, and coke production may be suppressed.

Further, the catalyst according to the present disclosure may be regenerated in a fluidized bed reactor, and the regenerated catalyst may be reused. Thus, process continuity may be increased and thus production of methyl acetate may be increased.

The polymer binder may be a material for binding the ferrierite zeolite catalyst particles to the surface of the support, and may be preferably a water-soluble polymer or a polymer having a functional group capable of binding to the ferrierite zeolite catalyst particles.

In one embodiment, the polymer binder may include at least one selected from polyethylene glycol (PEG), polyvinyl alcohol (PVA) and methyl cellulose. Specifically, a viscosity of the polyethylene glycol (PEG) may be preferably in a range of 114 to 142 mPa·s, a viscosity of the polyvinyl alcohol (PVA) may be preferably in a range of 35 to 55 mPa·s, and a viscosity of the methyl cellulose may be preferably in a range of 12 to 8000 cP.

In one embodiment, the polymer binder may be a polymer binder including a functional group including at least one selected from a methoxyl group (CH₃O—), a carboxyl group (—COOH), a glycerate (C₃H₅O₄—) and a hydroxyl group (—OH) as a functional group capable of binding to the ferrierite zeolite catalyst particles. When the polymer binder including the exemplified functional group is contained in the catalyst, the number of the ferrierite zeolite catalyst particles that may be bound to the support via the same amount of the polymer binder may be increased, thereby increasing the catalyst activity.

In one embodiment, a molecular weight of the polymer binder is preferably in a range of 146000 to 186000 g/mol. When the molecular weight of the polymer binder is in a range of 146000 to 186000 g/mol, as a chain length of the polymer binder increases, the number of the ferrierite zeolite catalyst particles that may be bound to the support via the same amount of the polymer binder may increase, thereby increasing the catalyst activity.

In one example, in the catalyst according to the present disclosure, a weight ratio between a content of the support and a content of the ferrierite zeolite catalyst particles is preferably in a range of 2.5:1 to 10:1. When the weight ratio (support/ferrierite zeolite catalyst particles) is smaller than 2.5:1, there is an excessive amount of ferrierite zeolite catalysts such that agglomeration occurs between the ferrierites rather than a uniform coating thereof occurs, thereby resulting in uneven coating. When the ratio exceeds 10:1, there is a problem in that an amount of the active material is too small and the catalyst reaction occurs insignificantly.

Further, in the catalyst according to the present disclosure, when the polymer binder is polyvinyl alcohol (PVA), a content of the polymer binder may be in a range of 9 to 73% by weight, preferably, of 50 to 60% by weight, based on a total weight of the support and the ferrierite zeolite catalyst particles. When the content of the polymer binder is smaller than 9% by weight based on the total weight of the support and the ferrierite zeolite catalyst particles, the ferrierite zeolite particles are not entirely coated on the support due to an insufficient amount of the binder relative to the catalyst and are partially removed from the support so that the catalyst activity is reduced. When the content exceeds 73 wt%, an exposed area of the ferrierite zeolite catalyst particles is reduced, so that the catalyst activity is reduced.

In another example, when the polymer binder is methyl cellulose, a content of the polymer binder is preferably in a range of 15 to 25% by weight of the total weight of the support and the ferrierite zeolite catalyst particles. When the content of the polymer binder is smaller than 15% by weight based on the total weight of the support and the ferrierite zeolite catalyst particles, the ferrierite zeolite particles are not entirely coated on the support due to an insufficient amount of the binder relative to the catalyst and are partially removed from the support so that the catalyst activity is reduced. When the content exceeds 25 wt %, an exposed area of the ferrierite zeolite catalyst particles is reduced, so that the catalyst activity is reduced.

As described above, in the catalyst according to the present disclosure, the content of each of the support, the ferrierite zeolite catalyst particles, and the polymer binder may be controlled to an appropriate range such that the ferrierite zeolite catalyst particles are uniformly coated on the surface of the support while maintains crystallinity thereof. Thus, the catalyst according to the present disclosure may exhibit high activity in a conversion reaction from dimethyl ether to methyl acetate, and the deactivated catalyst may be regenerated in the fluidized bed reactor and then reused.

Further, since the catalyst according to the present disclosure has the core-shell structure and is present in a form of a particle, deactivation thereof may be prevented, and coke deposition during the catalyst reaction may be suppressed, thereby enabling a stable process.

### Method for Preparing Catalyst for Carbonylation of Dimethyl Ether

A method for preparing a catalyst according to one embodiment of the present disclosure includes mixing a support having a first density, ferrierite zeolite catalyst particles having a second density smaller than the first density, and a polymer binder with each other in a solvent to prepare a mixture in S100, drying the mixture in S200, and firing the dried mixture in S300.

In one embodiment, S100 may include a first step S110 of mixing the support having a first density and the ferrierite zeolite catalyst particles having a second density smaller than the first density with each other, and a second step S120 of adding and mixing a mixture of the support and the ferrierite zeolite catalyst particles to and with a polymer binder solution.

The polymer binder solution in S120 may be prepared by dissolving the polymer binder in a solvent for 1 to 6 hours under conditions of pH 6 to 8 and 80 to 100° C. Under the above conditions, the polymer binder may be sufficiently dissolved in the solvent.

In S100, a weight ratio of the support and the ferrierite zeolite catalyst particles to be mixed with each other is preferably in a range of 2.5:1 to 10:1. When the weight ratio (support/ferrierite zeolite catalyst particles) is smaller than 2.5:1, there is an excessive amount of ferrierite zeolite catalysts such that agglomeration occurs between the ferrierites rather than a uniform coating thereof occurs, thereby resulting in uneven coating. When the ratio exceeds 10:1, there is a problem in that an amount of the active material is too small and the catalyst reaction occurs insignificantly.

Further, the content of the polymer binder to be added thereto is preferably in a range of 9 to 73% by weight of the total weight of the support and the ferrierite zeolite catalyst particles. When the content of the polymer binder is smaller than 9% by weight based on the total weight of the support and the ferrierite zeolite catalyst particles, the ferrierite zeolite particles are not entirely coated on the support due to an insufficient amount of the binder relative to the catalyst and are partially removed from the support so that the catalyst activity is reduced. When the content exceeds 73 wt %, an exposed area of the ferrierite zeolite catalyst particles is reduced, so that the catalyst activity is reduced.

In one embodiment, the polymer binder solution in which the polymer binder is dissolved in the solvent may be mixed with the support and the ferrierite zeolite catalyst particles. Alternatively, the polymer binder in a powder form may be mixed with the support and the ferrierite zeolite catalyst particles in a solvent. In this regard, the solvent is preferably distilled water. However, the present disclosure is not limited thereto.

In one example, the polymer binder preferably includes any at least one selected from polyethylene glycol (PEG), polyvinyl alcohol (PVA) and methyl cellulose. Specifically, the viscosity of the polyethylene glycol (PEG) is preferably in a range of 114 to 142 mPa·s, the viscosity of the polyvinyl alcohol (PVA) is preferably in a range of 35 to 55 mPa·s, and the viscosity of the methyl cellulose is preferably in a range of 12 to 8000 cP.

In one embodiment, when the polymer binder is polyethylene glycol (PEG) or polyvinyl alcohol (PVA), S300 may be performed at a temperature of 230 to 550° C. under a nitrogen atmosphere. Thus, a portion of the polymer binder contained in the dried mixture is decomposed and removed, and the remaining portion thereof remains. Thus, the catalyst in which a surface of each of at least some of the ferrierite zeolite catalyst particles bonded to the support surface is exposed to an outside may be prepared.

In one embodiment, when the polymer binder is methyl cellulose, S300 may include a first step of performing heat treatment at a temperature of 210 to 250° C. under a nitrogen atmosphere, a second step of performing heat treatment at a temperature of 230 to 270° C. under a nitrogen atmosphere after the first step, and a third step of performing heat-treatment at a temperature of 130 to 170° C. under a nitrogen atmosphere after the second step. Each of the first, second and third steps may be performed for 3 hours.

During the firing, a portion of the polymer binder contained in the dried mixture is decomposed and removed, and the remaining portion thereof remains, so that the catalyst in which the surface of each of at least some of the ferrierite zeolite catalyst particles bonded to the support surface is exposed to the outside may be prepared.

In one embodiment, the polymer binder may be a polymer binder including a functional group including at least one selected from a methoxyl group (CH₃O—), a carboxyl group (—COOH), a glycerate (C₃H₅O₄—) and a hydroxyl group (—OH) as a functional group capable of binding to the ferrierite zeolite catalyst particles. When the polymer binder including the exemplified functional group is contained in the catalyst, the number of the ferrierite zeolite catalyst particles that may be bound to the support via the same amount of the polymer binder may be increased, thereby increasing the catalyst activity.

In one embodiment, a molecular weight of the polymer binder is preferably in a range of 146000 to 186000 g/mol. When the molecular weight of the polymer binder is in a range of 146000 to 186000 g/mol, as a chain length of the polymer binder increases, the number of the ferrierite zeolite catalyst particles that may be bound to the support via the same amount of the polymer binder may increase, thereby increasing the catalyst activity.

According to the method for preparing the catalyst according to the present disclosure, during the preparation, the content of each of the support, the ferrierite zeolite catalyst particles, and the polymer binder may be controlled to an appropriate range such that the ferrierite zeolite catalyst particles may be successfully bound to the surface of the support, and a portion of the polymer binder may remain via the firing process. Thus, the catalyst may be prepared in which the surface of each of at least some of the ferrierite zeolite catalyst particles bound to the support surface is exposed to the outside.

The method for preparing the catalyst according to the present disclosure is economical and has a fast synthesis rate because the method does not use a special mechanical apparatus, which is not the case in a conventional method for preparing a catalyst for a fluidized bed in which a density is controlled using extrusion, compression, and spraying.

### Method for Preparing Methyl Acetate

A method for preparing methyl acetate according to one embodiment of the present disclosure may include performing carbonylation of dimethyl ether using carbon monoxide under presence of the catalyst according to the present disclosure, thereby converting dimethyl ether to methyl acetate.

In one embodiment, converting dimethyl ether to methyl acetate may include reacting a mixed gas containing carbon monoxide and dimethyl ether at a temperature of 200 to 240° C. in a fluidized bed reactor. For example, the mixed gas may be a mixed gas containing carbon monoxide: dimethyl ether: nitrogen at 4.5:90:5.5 mol %.

Further, before the carbonylation, a pretreatment process of removing foreign substances such as water molecules present in the catalyst according to the present disclosure may be performed by raising a temperature of the catalyst to 500° C. under a nitrogen gas atmosphere and then maintaining the catalyst for 1 hour.

In one example, the carbonylation may be performed for 15 to 19 hours at a reaction temperature of 200 to 240° C. and a pressure of 10 to 30 bar. When the reaction is carried out within the temperature and pressure ranges, a reaction rate and selectivity of methyl acetate may be improved.

### Method for Regenerating Catalyst

According to the present disclosure, after the carbonylation, a deactivated catalyst is injected into a fluidized bed reactor, and a carbon monoxide-containing gas is added thereto to regenerate the catalyst. Since the catalyst according to the present disclosure includes the support having a density set such that the support can flow in the fluidized bed reactor, the catalyst may be regenerated while the support flows in the fluidized bed reactor.

Hereinafter, preparation and characteristics of the catalyst according to the present disclosure will be described in more detail based on specific Examples.

### EXAMPLES

### Preparation of Catalyst for Carbonylation of Dimethyl Ether

### Present Example 1: Preparation of FCC@CFER=10:1 (PVA, 54.5%) Catalyst

10 g of support (commercial fluidized bed (FCC) catalyst) and 1 g of commercial ferrierite zeolite catalyst particles were mixed with each other via physical agitation.

Further, 6 g of PVA having a viscosity of 35 to 55 mPa·s was sufficiently dissolved in distilled water at pH 6 to 8 and 80 to 100° C. for 1 hour to prepare a polymer binder solution.

Thereafter, a mixture of the support and the ferrierite zeolite catalyst particles was added to the polymer binder solution, and stirring of the mixed solution was executed for 6 hours. The resulting mixed solution was dried in an oven at 110° C. for 24 hours.

Next, the dried mixture was fired at 550° C. for 3 hours under a nitrogen atmosphere to prepare the catalyst (Present Example 1, FCC@CFER=10:1 (PVA, 54.5%)) according to the present disclosure).

### Present Examples 2 to 3

The same process as the preparation process of the catalyst according to Present Example 1 was performed except that each of 9.167 g and 7.857 g of the support was used, and the weight ratio of the support and the ferrierite zeolite catalyst particles was changed to each of 5:1 and 2.5:1. Thus, we obtained a catalyst of FCC@CFER=5:1 (PVA, 54.5%) in Present Example 2 and a catalyst of FCC@CFER=2.5:1 (PVA, 54.5%) in Present Example 3.

### Present Examples 4 to 5

The same process as the preparation process of the catalyst according to Present Example 1 was performed except that PEG was used as the polymer binder. Thus, a catalyst of Present Example 4 (FCC@CFER=10:1 (PEG, 54.5%) was obtained.

Further, the same process as the preparation process of the catalyst according to Present Example 3 was performed except that PEG was used as the polymer binder. Thus, a catalyst of Present Example 5 FCC@CFER=2.5:1 (PEG, 54.5%) was obtained.

### Present Example 6: Preparation of Layer/FCC@CFER=10:1 (Methyl Cellulose, 36%) Catalyst

10 g of support (commercial fluidized bed (FCC) catalyst) and 1 g of commercial ferrierite zeolite catalyst particles were mixed with each other via physical agitation.

Then, 4 g of methyl cellulose having a viscosity of 15 cp and a mixture of the support and the ferrierite zeolite catalyst particles were sieved and laminated with each other, and a resulting mixture was dried in an oven at 110° C. for 24 hours.

Next, the dried mixture was fired for 3 hours at a temperature at each of 230° C., 250° C. and 150° C. under a nitrogen atmosphere, and thus a catalyst according to the present disclosure (Present Example 6, Layer/FCC@CFER=10:1 (Methyl cellulose), 36%)) was prepared.

### Present Examples 7 to 8

The same process as the preparation process of the catalyst according to Present Example 6 was performed except that each of 9.167 g and 7.857 g of the support was used, and the weight ratio of the support and the ferrierite zeolite catalyst particles was changed to each of 5:1 and 2.5:1. Thus, a catalyst of Present Example 7 (Layer/FCC@CFER=5:1 (Methyl cellulose, 36%)), and a catalyst of Present Example 8 (Layer/FCC@CFER=2.5:1 (Methyl cellulose, 36%)) were obtained.

### Present Example 9: Preparation of FCC@CFER=10:1 (Methyl Cellulose, 36%) Catalyst

10 g of the support (commercial fluidized bed (FCC) catalyst) and 1 g of commercial ferrierite zeolite catalyst particles were mixed with each other via physical agitation.

Further, 4 g of methyl cellulose having a viscosity of 15 cp was sufficiently dissolved in distilled water at pH 6 to 8 and 80 to 100° C. for 1 hour to prepare a polymer binder solution.

Thereafter, a mixture of the support and the ferrierite zeolite catalyst particles was added to the polymer binder solution, and stirring of the mixed solution was continued for 6 hours. The resulting mixed solution was dried in an oven at 110° C. for 24 hours.

Next, the dried mixture was fired for 3 hours at a temperature at each of 230° C., 250° C. and 150° C. under a nitrogen atmosphere, and thus a catalyst according to the present disclosure (Present Example 9, FCC@CFER=10:1 (Methyl cellulose, 36%)) was prepared.

### Present Examples 10 to 11

The same process as the preparation process of the catalyst according to Present Example 9 was performed except that each of 9.167 g and 7.857 g of the support was used, and a weight ratio of the support and the ferrierite zeolite catalyst particles was changed to each of 5:1 and 2.5:1. Thus , a catalyst (FCC@CFER=5:1 (Methyl cellulose, 36%)) of Present Example 10 and a catalyst (FCC@CFER=2.5:1 (Methyl cellulose, 36%)) of Present Example 11 were obtained.

### Present Examples 12 to 14

The same process as the preparation process of the catalyst according to Present Example 9 was performed except that instead of the commercial ferrierite zeolite catalyst particles, H-form ferrierite zeolite catalyst particles (NSFER (0.2), where 0.2 denotes an amount of nanosheet ferrierite as a seed) synthesized using seed synthesis and subjected to ion exchange were used, and 5 g of the support (commercial fluidized bed (FCC) catalyst) and 0.5 g of nanosheet ferrierite were used. Thus, a catalyst (FCC@NSFER (0.2)=10:1 (Methyl cellulose, 73%)) of Present Example 12 was obtained.

Further, the same process as the preparation process of the catalyst according to Present Example 12 was performed except that 4.58 g and 3.93 g of the support and 0.917 g and 1.57 g of the nanosheet ferrierite were used, respectively. Thus, a catalyst (FCC@NSFER(0.2)=5:1 (Methyl cellulose, 73%)) of Present Example 13, and a catalyst of FCC@NSFER (0.2)=2.5:1 (Methyl cellulose, 73%)) of Present Example 14 were obtained.

### Present Example 15

The same process as the preparation process of the catalyst according to Present Example 3 was performed except that 8.8 g of PVA was used as the polymer binder. Thus, a catalyst (FCC@CFER=2.5:1 (PVA, 80%)) of Present Example 15 was obtained.

### Present Example 16

A commercial ferrierite zeolite catalyst particle (VFER) was prepared.

### Present Example 17

H-form ferrierite zeolite catalyst particles (NSFER (0.2), where 0.2 denotes an amount of nanosheet ferrierite as a seed) synthesized using seed synthesis and subjected to ion exchange were prepared.

### Present Example 18: Preparation of FCC@CFER=2.5:1 (Methyl Cellulose, 18%, 130-150-50) Catalyst

A dried mixture was obtained in the same manner as in Present Example 11 except that 2 g of methyl cellulose having a viscosity of 8000 cp was used. Then, the dried mixture was fired for 3 hours at a temperature of each of 130° C., 150° C. and 50° C. under a nitrogen atmosphere. Thus, a catalyst according to the present disclosure (Present Example 18, FCC@CFER=2.5:1 (Methyl cellulose, 18%), 130-150-50)) was prepared.

### Present Example 19

The same process as the preparation process of the catalyst according to Present Example 18 was performed except that the firing temperature was changed to 430° C., 450° C. and 350° C. Thus, a catalyst (FCC@CFER=2.5:1 of (Methyl cellulose, 18%, 430-450-350)) of Present Example 19 was obtained.

### Present Examples 20 to 23

The same process as the preparation process of the catalyst according to Present Example 1 was performed except that PVA having a viscosity of 35 to 55 mPa·s was used at a content of each of 9 wt %, 27 wt %, 54.5 wt %, and 90 wt %, based on a total weight of the support and the ferrierite zeolite catalyst particles. Thus, a catalyst (FCC@CFER=10:1 (PVA, 9%, 550)) of Present Example 20, a catalyst (FCC@CFER=10:1 (PVA, 27%, 550)) of Present Example 21, a catalyst (FCC@CFER=10:1 (PVA, 54.5%, 550)) of Present Example 22, and a catalyst (FCC@CFER=10:1 (PVA, 90%, 550)) of Present Example 23 were obtained.

### Present Example 24: Preparation of FCC@CFER=5:1 (PVA,18%,250) Catalyst

A dried mixture was prepared in the same manner as in Present Example 2, except that PVA having a viscosity of 11.6 to 15.4 mPa·s was used at a content of 18% by weight, based on a total weight of the support and the ferrierite zeolite catalyst particles. Thereafter, the dried mixture was fired at 250° C. for 3 hours under a nitrogen atmosphere to prepare a catalyst (Present Example 24, FCC@CFER=5:1 (PVA, 18%, 250)) according to the present disclosure.

### Present Examples 25 to 26

The same process as the preparation process of the catalyst according to Present Example 24 was performed except that the firing temperature was changed to 650° C. and 750° C. Thus, a catalyst (FCC@CFER=5:1 (PVA, 18%, 650)) of Present Example 26, and a catalyst (FCC@CFER=5:1 (PVA, 18%, 750)) of Present Example 26 were prepared.

### Comparative Example 1

A commercial fluidized bed (FCC) catalyst was prepared.

### Structure Analysis of Catalyst

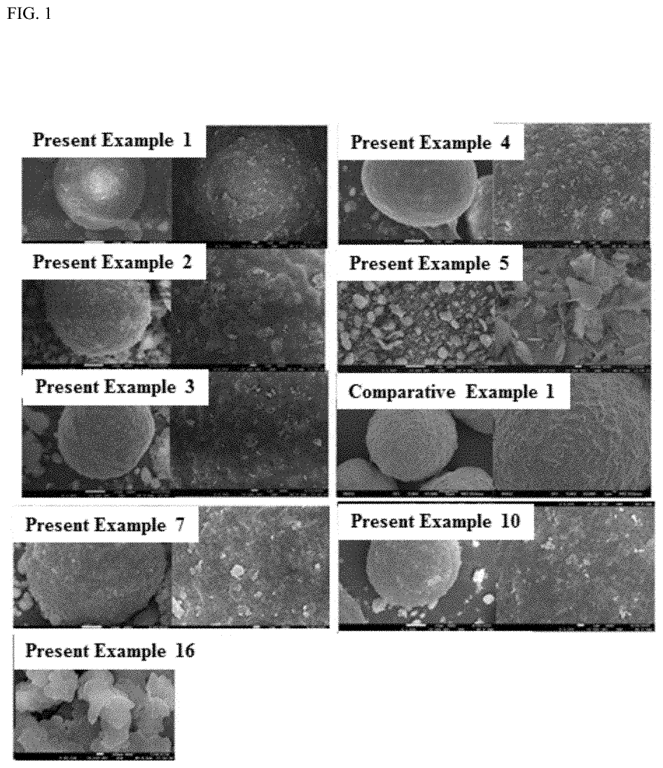

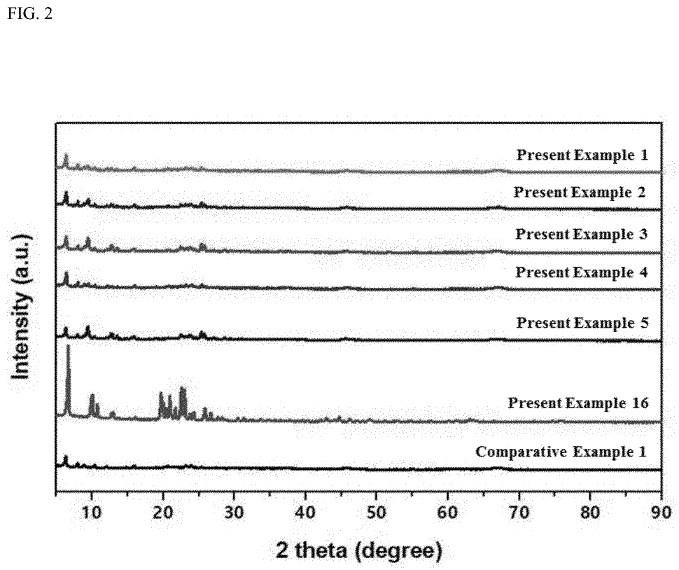

SEM analysis and X-ray diffraction analysis were performed to identify structures of catalysts prepared according to Present Examples and Comparative Examples of the present disclosure. Results thereof are shown in FIGS. 1 and 2, respectively.

Referring to FIG. 1, it may be identified that in each of the catalysts (Present Examples 1 to 5, Present Example 7, and Present Example 10) according to Present Examples of the present disclosure, the ferrierite zeolite catalyst particles are present on the surface of the support. Further, it may be observed that each of the catalysts (Present Examples 1 to 5, Present Example 7, and Present Example 10) according to Present Examples of the present disclosure has a core-shell structure in which the ferrierite zeolite catalyst particles are coated on the support surface, and an intrinsic structure of the ferrierite is not changed.

Further, referring to FIG. 2 as the result of X-ray diffraction analysis, it may be identified that in the catalyst according to each of Present Examples 1 to 5, an intrinsic skeletal structure of the ferrierite is maintained.

### Analysis of Acid Site of Catalyst

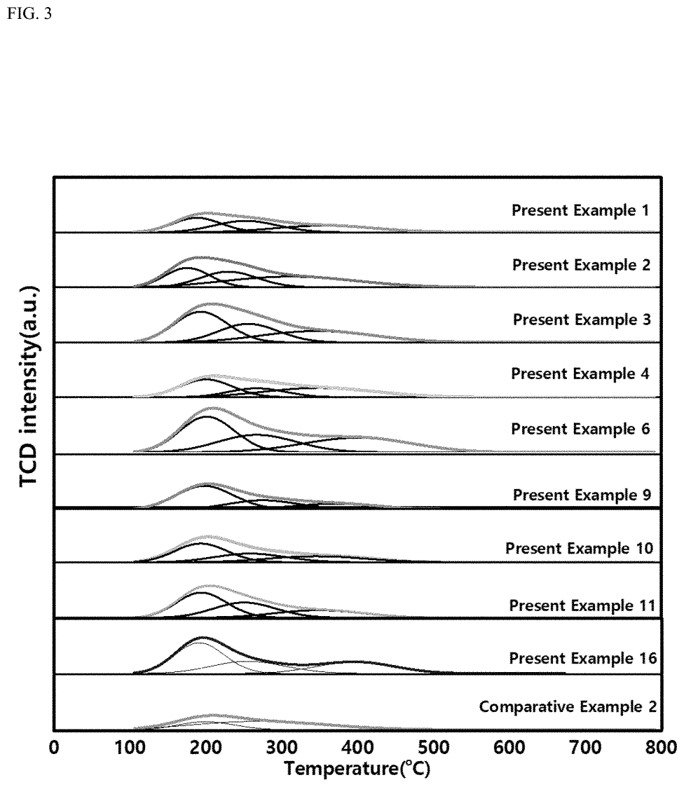

In order to analyze an acid site of each of the catalysts prepared according to the Present examples of the present disclosure, TPD analysis (NH₃-TPD) was performed using ammonia as a basic material, and results are shown in FIG. 3 and Table 1. In this regard, a peak corresponding to a strong acid in the TPD analysis result is classified as a Brønsted acid site, while the remaining acid site is classified as a Lewis acid site or defect site.

TABLE 1

| | | | M(220 | S(400 |
|---|---|---|---|---|
| | | | to | to |
| | | | 400° C.) | 485° C.) |
| | | W(~220° C.) | (mmol/ | (mmol/ |
| Examples | Zeolite catalyst | (mmol/gcat) | gcat) | gcat) |
| Present | FCC@CFER = 10:1 | 0.072 | 0.072 | 0.064 |
| Example 1 | (PVA, 54.5%) | | | |
| Present | FCC@CFER = 5:1 | 0.09 | 0.088 | 0.134 |
| Example 2 | (PVA, 54.5%) | | | |
| Present | FCC@CFER = 2.5:1 | 0.165 | 0.108 | 0.121 |
| Example 3 | (PVA, 54.5%) | | | |
| Present | FCC@CFER = 10:1 | 0.094 | 0.049 | 0.098 |
| Example 4 | (PEG, 54.5%) | | | |
| Present | FCC@CFER = 2.5:1 | 0.195 | 0.134 | 0.148 |
| Example 5 | (PEG, 54.5%) | | | |
| Present | FCC@CFER = 10:1 | 0.133 | 0.058 | 0.042 |
| Example 9 | (Methyl cellulose, 36%) | | | |
| Present | FCC@CFER = 5:1 | 0.098 | 0.059 | 0.053 |
| Example 10 | (Methyl cellulose, 36%) | | | |
| Present | FCC@CFER = 2.5:1 | 0.127 | 0.092 | 0.062 |
| Example 11 | (Methyl cellulose, 36%) | | | |
| Present | VFER | 0.757 | 0.199 | 0.163 |
| Example16 | | | | |
| Comparative | FCC | 0.028 | 0.064 | — |
| Example 3 | | | | |

Referring to Table 1, it may be identified that the FCC catalyst as the support has no acid site, and thus has no activity in the reaction. It may be observed that as the content of the ferrierite zeolite coated on the support increases, the number of acid sites increases. Therefore, the number of acid sites may be adjusted based on an amount of the coating, and thus, the reactivity or activity may also be selectively changed.

### Carbonylation of Dimethyl Ether

### Present Examples 1-1 to 1-15, and Present Examples 1-18 to 1-26

A pretreatment process in which each of the catalysts prepared according to Present Examples 1 to 15 and Present Examples 18 to 26 was heated to 500° C. under a nitrogen gas atmosphere and maintained for 1 hour to remove foreign substances such as water molecules present in the catalyst was performed. After the pretreatment, 2 g of catalyst was introduced into the reactor, and a mixed gas containing dimethyl ether:carbon monoxide:nitrogen at 4.5:90:5.5 mol % was injected into the reactor and flowed therein at a reaction pressure of 20 kg/cm³, a space velocity of 500 L/kgcat/h, and a reaction temperature of 220° C. Thus, carbonylation was carried out for 17 hours.

### Present Examples 1-16 and 1-17, and Comparative Example 1-1

A pretreatment process in which each of the catalysts of Present Examples 16 to 17 and Comparative Example 1 was heated to 500° C. under a nitrogen gas atmosphere and maintained for 1 hour to remove foreign substances such as water molecules present in the catalyst was performed. After the pretreatment, 0.4 g of catalyst was introduced into the reactor, and a mixed gas containing dimethyl ether:carbon monoxide:nitrogen at 4.5:90:5.5 mol % was injected into the reactor and flowed therein at a reaction pressure of 20 kg/cm3, a space velocity of 2000 L/kgcat/h, and a temperature of 220° C. Thus, carbonylation was carried out for 17 hours.

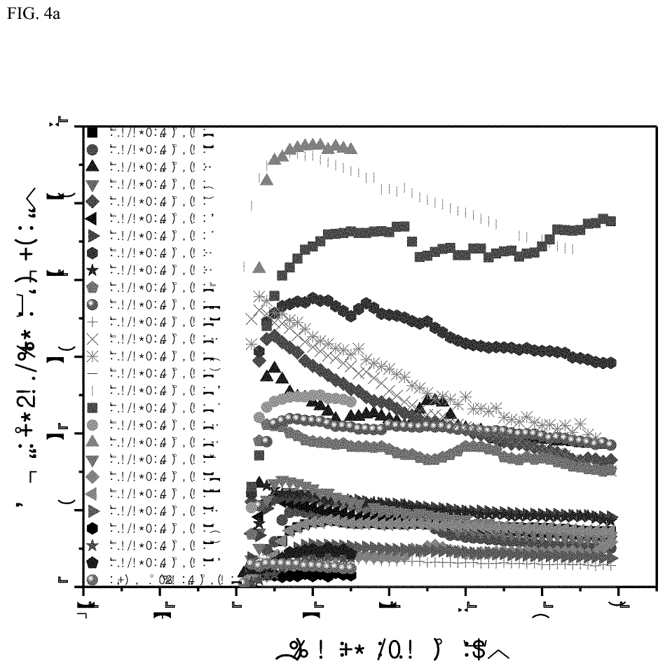

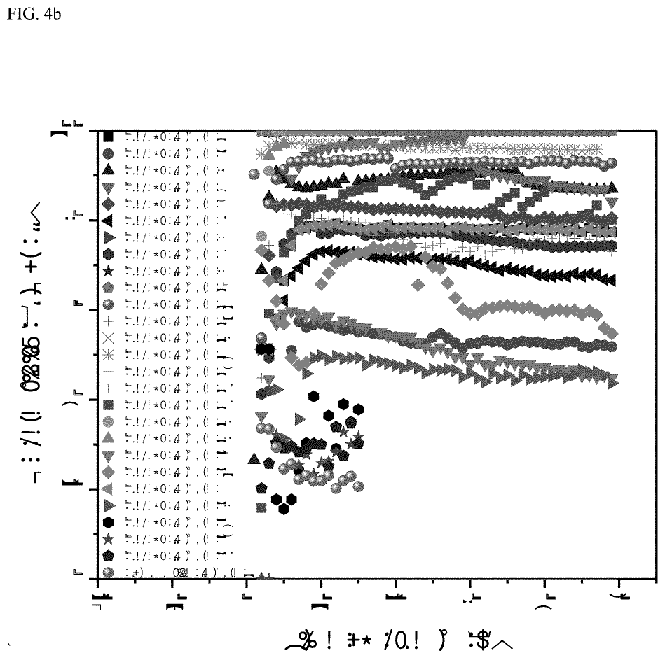

A composition of a product from the reaction was analyzed using gas chromatography. Dimethyl ether conversion (mol %), methyl acetate selectivity (mol %), and deactivation rate (mol %/h) were calculated using the analysis results. The deactivation rate is defined as an average change rate from a time corresponding to a maximum conversion rate to a last time. Specific reaction results are shown in Table 2 and FIG. 4A and FIG. 4B.

TABLE 2

| | | Con- | Selectivity | Deactiv- |
|---|---|---|---|---|
| | | version | [MA/ | ation |
| | | [DME] | MeOH] | rate |
| Examples | Zeolite catalyst | (Mol %) | (Mol %) | (Mol %/h) |
| Present | FCC@CFER = | 4.3 | 79.2 | 0.02 |
| Example | 10:1(PVA, 54.5%) | | | |
| 1-1 | | | | |
| Present | FCC@CFER = | 5.0 | 57.4 | 0.06 |
| Example | 5:1 (PVA, 54.5%) | | | |
| 1-2 | | | | |
| Present | FCC@CFER = | 14.2 | 97.6 | 0.12 |
| Example | 2.5:1(PVA, 54.5%) | | | |
| 1-3 | | | | |
| Present | FCC@CFER = | 6.2 | 61.8 | 0.07 |
| Example | 10:1 (PEG, 54.5%) | | | |
| 1-4 | | | | |
| Present | FCC@CFER = | 16.4 | 83.7 | 0.18 |
| Example | 2.5:1(PEG, 54.5%) | | | |
| 1-5 | | | | |
| Present | layer/FCC@CFER = | 5.8 | 73 | 0.05 |
| Example | 10:1 (Methyl | | | |
| 1-6 | cellulose, 36%) | | | |
| Present | Layer/FCC@CFER = 5:1 | 5.9 | 79 | 0.04 |
| Example | | | | |
| 1-7 | (Methyl cellulose, 36%) | | | |
| Present | Layer/FCC@CFER = 2.5:1 | 18.9 | 100 | 0.1 |
| Example | (Methyl cellulose, 36%) | | | |
| 1-8 | | | | |
| Present | FCC@CFER = 10:1 | 6.6 | 100.0 | 0.05 |
| Example | (Methyl cellulose, 36%) | | | |
| 1-9 | | | | |
| Present | FCC@CFER = 5:1 | 10.6 | 100.0 | 0.07 |
| Example | (Methyl cellulose, 36%) | | | |
| 1-10 | | | | |
| Present | FCC@CFER = 2.5:1 | 11.0 | 94.0 | 0.04 |
| Example | (Methyl cellulose, 36%) | | | |
| 1-11 | | | | |
| Present | FCC@NSFER(0.2) = 10:1 | 2.1 | 83.9 | 0.01 |
| Example | (Methyl cellulose, 73%) | | | |
| 1-12 | | | | |
| Present | FCC@NSFER(0.2) = 5:1 | 18.0 | 100.0 | 0.22 |
| Example | (Methyl cellulose, 73%) | | | |
| 1-13 | | | | |
| Present | FCC@NSFER(0.2) = 2.5:1 | 18.9 | 97.3 | 0.2 |
| Example | (Methyl cellulose, 73%) | | | |
| 1-14 | | | | |
| Present | FCC@CFER = 2.5:1 | 0 | 0 | 0 |
| Example | (PVA, 80%) | | | |
| 1-15 | | | | |
| Present | VFER | 28.3 | 100 | 0.17 |
| Example | | | | |
| 1-16 | | | | |
| Present | NSFER(0.2) | 24.0 | 89.9 | 0.45 |
| Example | | | | |
| 1-17 | | | | |
| Present | FCC@CFER = 2.5:1 | 12.5 | 100 | 0.09 |
| Example | (Methyl cellulose, 18%, | | | |
| 1-18 | 130-150-50) | | | |
| Present | FCC@CFER = | 28.8 | 100 | 0.6 |
| Example | 2.5:1 (Methyl | | | |
| 1-19 | cellulose,18%, | | | |
| | 430-450-350) | | | |
| Present | FCC@CFER = | 7.0 | 59.7 | 0.08 |
| Example | 10:1(PVA, 9%, 550) | | | |
| 1-20 | | | | |
| Present | FCC@CFER = | 3.1 | 72.3 | 0 |
| Example | 10:1(PVA, 27%, 550) | | | |
| 1-21 | | | | |
| Present | FCC@CFER = | 4.3 | 79.2 | 0.02 |
| Example | 10:1(PVA, 54.5%, 550) | | | |
| 1-22 | | | | |
| Present | FCC@CFER = | 2.8 | 64.2 | 0.02 |
| Example | 10:1(PVA, 90%, 550) | | | |
| 1-23 | | | | |
| Present | FCC@CFER = | 0.9 | 37.8 | 0.08 |
| Example | 5:1(PVA, 18%, 250) | | | |
| 1-24 | | | | |
| Present | FCC@CFER = | 2.1 | 32.8 | 0.08 |
| Example | 5:1(PVA, 18%, 650) | | | |
| 1-25 | | | | |
| Present | FCC@CFER = | 2.5 | 34.75 | 0.1 |
| Example | 5:1(PVA, 18%, 750) | | | |
| 1-26 | | | | |
| Com- | FCC | 1.5 | 33.6 | 0.02 |
| parative | | | | |
| Example | | | | |
| 1-1 | | | | |

### Catalyst Activity Based on Content of Each of Support, Ferrierite Zeolite Catalyst Particles, and Polymer Binder

Referring to Table 2, it may be identified that the dimethyl ether conversion of each of the catalysts of Present Examples 1-1 to 1-14 varies based on the weight content of the added ferrierite zeolite catalyst particles. As described above, the reactivity or activity of the catalyst may be controlled by appropriately adjusting the content of each of the support, the ferrierite zeolite catalyst particles, and the polymer binder.

To the contrary, in Present Example 1-15 where the content of the polymer binder exceeds 73% by weight based on the total weight of the support and the ferrierite zeolite catalyst particles, the ferrierite zeolite catalyst particles may not bind to the support surface, resulting in no catalyst reactivity or activity.

Therefore, in order for the catalyst according to the present disclosure to exhibit high activity, the weight ratio of the support and the ferrierite zeolite catalyst particles may be in a range of 2.5:1 to 10:1, and the content of the polymer binder may be in a range of 9 to 73% by weight based on the total weight of the support and the ferrierite zeolite catalyst particles.

Further, it may be identified from Tables 1 and 2 that as an amount of the ferrierite zeolite catalyst particles coated on the support surface increases while the above defined content range is satisfied, the number of the acid sites that play an important role in DME carbonylation may increase, resulting in high reactivity or activity.

### Optimal Content of Polymer Binder Remaining in Entirety of Catalyst

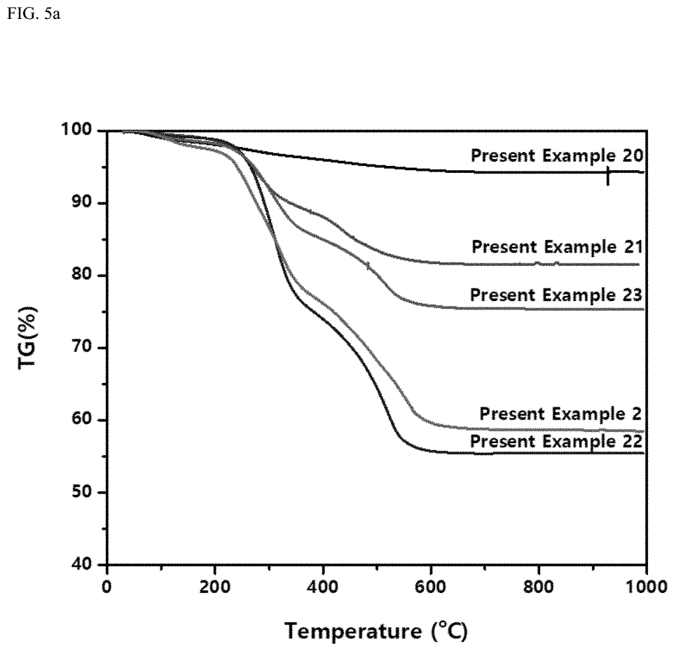

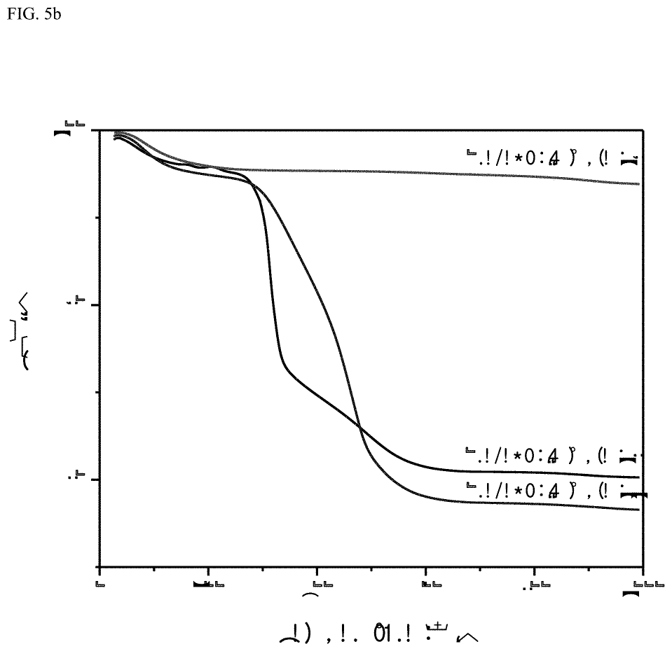

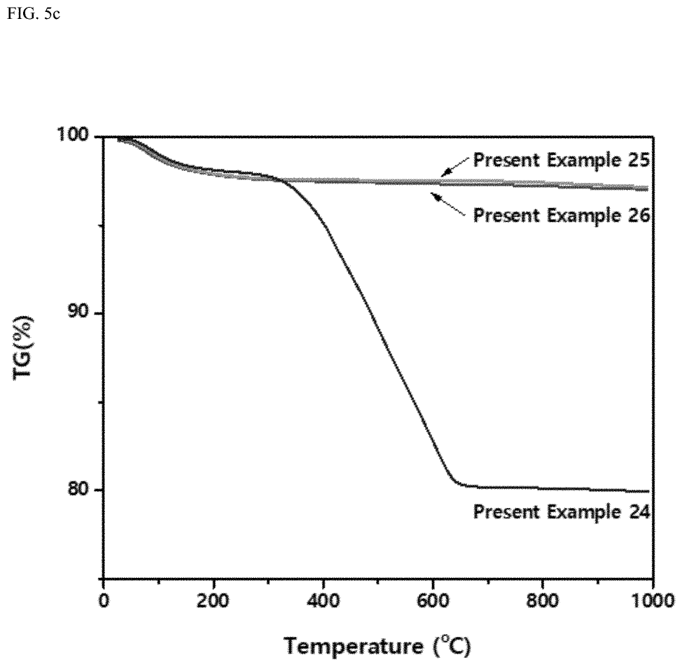

Using the Table 2 and FIGS. 5A to 5C showing the results of TGA analysis, a content of the polymer binder remaining in the catalyst after the firing was identified.

First, it may be identified that in a catalyst of Present Example 2 and a catalyst of each of Present Examples 20 to 23, when the content of the added binder is 54 wt %, 50 to 60% of the polymer binder remains after the firing at an optimum temperature, and in this case, the reactivity or activity of the catalyst has a maximal level. This is because, when the content of the residual binder is too high, the exposed area of the ferrierite particles is reduced and thus the catalyst activity decreases, whereas when the content of the residual binder is too low, the catalyst activity decreases because the number of ferrierite particles bound to the support decreases.

Further, it may be identified that in Present Examples 6 to Present Example 14 using the methyl cellulose polymer binder, the reactivity or activity of the catalyst is excellent when the content of the remaining polymer binder is about 20%.

### Catalyst Activity Based on Viscosity (Molecular Weight) of Polymer Binder

Using Table 2 and FIGS. 5A to 5C showing the results of TGA analysis, the catalyst activity based on a viscosity of the polymer binder was identified.

First, each of the catalysts of Present Examples 24 to 26 using PVA with a short polymer chain exhibits lower reactivity or activity than that of each of the catalysts of Present Example 2 and Present Examples 20 to 23 using PVA with a longer polymer chain.

Further, when methyl cellulose is used as the polymer binder, the catalyst of each of Present Examples 18 to 19 having methyl cellulose having a long polymer chain exhibits better reactivity or activity, compared to the catalyst of each of Examples 6 to 14 having methyl cellulose having a short polymer chain.

This is because as the chain length of the polymer binder increases (that is, as the viscosity (molecular weight) thereof increases), the number of ferrierite particles that may be bound to the support at the same amount of the binder may increase.

A scope of protection of the present disclosure should be construed by the scope of the claims, and all technical ideas within the scope equivalent thereto should be construed as being included in the scope of the present disclosure. Although the embodiments of the present disclosure have been described in more detail with reference to the accompanying drawings, the present disclosure is not necessarily limited to these embodiments. The present disclosure may be implemented in various modified manners within the scope not departing from the technical idea of the present disclosure. Accordingly, the embodiments disclosed in the present disclosure are not intended to limit the technical idea of the present disclosure, but to describe the present disclosure. the scope of the technical idea of the present disclosure is not limited by the embodiments. Therefore, it should be understood that the embodiments as described above are illustrative and non-limiting in all respects. The scope of protection of the present disclosure should be interpreted by the claims, and all technical ideas within the scope of the present disclosure should be interpreted as being included in the scope of the present disclosure.

What is claimed is:

1. A catalyst for carbonylation of dimethyl ether, the catalyst comprising:
a support having a first density; and
ferrierite zeolite catalyst particles bound to a surface of the support via a polymer binder and having a second density smaller than the first density.

2. The catalyst of claim 1, wherein a surface of each of at least some of the ferrierite zeolite catalyst particles is exposed to an outside.

3. The catalyst of claim 1, wherein the catalyst has a core-shell structure in which the ferrierite zeolite catalyst particles are coated on the surface of the support.

4. The catalyst of claim 1, wherein a ratio between a weight of the support and a weight of the ferrierite zeolite catalyst particles is in a range of 2.5:1 to 10:1.

5. The catalyst of claim 1, wherein the polymer binder includes at least one selected from a group consisting of polyethylene glycol (PEG), polyvinyl alcohol (PVA) and methyl cellulose.

6. The catalyst of claim 5, wherein when the polymer binder is polyvinyl alcohol (PVA), a content of the polymer binder is in a range of 9 to 73% by weight, based on a total weight of the support and the ferrierite zeolite catalyst particles.

7. The catalyst of claim 5, wherein when the polymer binder is methyl cellulose, a content of the polymer binder is a range of 15 to 25% by weight, based on a total weight of the support and the ferrierite zeolite catalyst particles.

8. The catalyst of claim 1, wherein the polymer binder has at least one functional group selected from a group consisting of a methoxyl group (CH₃O—), a carboxyl group (—COOH), glycerate (C₃H₅O₄—) and a hydroxyl group (—OH).

9. The catalyst of claim 1, wherein a molecular weight of the polymer binder is in a range of 146000 to 186000 g/mol.

10. The catalyst of claim 1, wherein a molar ratio (Si/Al) as a ratio of a content of silicon to a content of aluminum in the ferrierite zeolite catalyst particle is in a range of 5 to 30.

11. The catalyst of claim 1, wherein a size of the support is in a range of 50 to 150 μm, wherein a size of each of the ferrierite zeolite catalyst particles is in a range of 100 nm to 1 μm.

12. The catalyst of claim 1, wherein the first density is in a range of 750 to 800 kg/m³.

13. A method for preparing a catalyst for carbonylation of dimethyl ether, the method comprising:
mixing a support having a first density, ferrierite zeolite catalyst particles having a second density smaller than the first density, and a polymer binder with each other in a solvent to prepare a mixed solution;
drying the mixed solution to prepare a dried mixture; and
firing the dried mixture.

14. The method of claim 13, wherein in the firing of the dried mixture, a portion of the polymer binder contained in the dried mixture is decomposed and removed, and the rest thereof remains therein such that a surface of each of at least some of the ferrierite zeolite catalyst particles bound to a surface of the support is exposed to an outside.

15. The method of claim 13, wherein the mixing includes:
a first step of mixing the support having the first density and the ferrierite zeolite catalyst particles having the second density smaller than the first density with each other to prepare a mixture; and
a second step of adding and mixing the mixture of the support and the ferrierite zeolite catalyst particles to and with a polymer binder solution.

16. The method of claim 15, wherein the polymer binder solution is prepared by dissolving the polymer binder in a solvent for 1 to 6 hours under conditions of pH 6 to 8, and 80 to 100° C.

17. The method of claim 13, wherein in the mixing, a ratio between a weight of the support and a weight of the ferrierite zeolite catalyst particles is in a range of 2.5:1 to 10:1.

18. The method of claim 13, wherein in the mixing, a content of the polymer binder is in a range of 9 to 73% by weight, based on a total weight of the support and the ferrierite zeolite catalyst particles.

19. The method of claim 13, wherein the polymer binder includes at least one selected from a group consisting of polyethylene glycol (PEG), polyvinyl alcohol (PVA) and methyl cellulose.

20. The method of claim 19, wherein when the polymer binder is polyethylene glycol (PEG) or polyvinyl alcohol (PVA), the firing is performed at a temperature of 230 to 550° C. under a nitrogen atmosphere.

21. The method of claim 19, wherein when the polymer binder is methyl cellulose, the firing includes:
a first step of performing heat treatment at a temperature of 210 to 250° C. under a nitrogen atmosphere;
a second step of performing heat-treatment at a temperature of 230 to 270° C. under a nitrogen atmosphere after the first step; and
a third step of performing heat treatment at a temperature of 130 to 170° C. under a nitrogen atmosphere after the second step.

22. The method of claim 13, wherein the polymer binder has at least one functional group selected from a group consisting of a methoxyl group (CH₃O—), a carboxyl group (—COOH), glycerate (C₃H₅O₄—) and a hydroxyl group (—OH).

23. The method of claim 13, wherein a molecular weight of the polymer binder is in a range of 146000 to 186000 g/mol.

24. A method for preparation of methyl acetate, the method comprising performing carbonylation of dimethyl ether using carbon monoxide under presence of the catalyst of claim 1, thereby converting dimethyl ether to methyl acetate.

25. The method of claim 24, wherein the performing of the carbonylation of dimethyl ether using carbon monoxide under presence of the catalyst, thereby converting dimethyl ether to methyl acetate includes reacting a mixed gas containing carbon monoxide and dimethyl ether in a fluidized bed reactor at a temperature of 200 to 240° C.

26. A method for regenerating a catalyst, the method comprising treating a deactivated catalyst with carbon monoxide-containing gas in a fluidized bed reactor, thereby regenerating the catalyst, wherein the catalyst includes the catalyst of claim 1.
